(19) United States
(12) Patent Application Publication
(10) Pub. No.: US 2023/0398344 A1
(43) Pub. Date: Dec. 14, 2023

(54) SEALING DEVICE FOR NEEDLELESS CONNECTORS

(71) Applicant: Quest Medical, Inc., Allen, TX (US)

(72) Inventors: Jeffrey Albertsen, McKinney, TX (US); Robert Mart, Colleyville, TX (US); Andrew Nelson, Dallas, TX (US); James Bernal, Frisco, TX (US)

(21) Appl. No.: 17/837,236

(22) Filed: Jun. 10, 2022

(51) Int. Cl. A61M 39/20; A61M 39/16; B29C 70/76

(52) U.S. Cl. CPC A61M 39/20; A61M 39/162; B29C 70/76; A61M 2207/00; B29L 2031/26

(57) ABSTRACT

A sealing device for sealing an opening of an apparatus. The sealing device has a head that defines a cavity and a neck that defines a passage disposed between the cavity at a first end of the neck and an aperture at a second end of the neck. The aperture is configured to collapse and expand, having a first diameter in a collapsed configuration and a second diameter in an expanded configuration. The interior surface of the neck includes an engagement interface that is configured to releasably engage with a spacer. When the engagement interface is engaged with the spacer, the neck is in the expanded configuration.

### BACKGROUND

### Technical Field

Novel aspects of the present disclosure relate to the field of medical devices. More particularly, and not by way of limitation, the present disclosure is directed to a single use cap for connectors.

### Background

Needleless connectors (NCs) are transitional interfaces that permit the exchange of fluids between containers, fluid transfer devices, and/or fluid conduits. NCs were designed primarily for use in the medical field to prevent needlestick injuries to medical care professionals. The NCs are typically attached to the ends of vascular catheters or other tubular structures, such as branched intravenous (IV) lines, to facilitate access for infusion and aspiration without the need for needles.

NCs generally have a housing that define a fluid pathway between a distal end to a proximal end. For some NCs, the proximal end is configured to engage with a fluid distribution line, such as a catheter or IV, and the distal end is exposed to the environment and configured to engage with fluid transfer device, such as a syringe, or fluid sources, such as a vial. For other NCs, the proximal end is configured to engage with a fluid source, such as a vial, and the distal end is exposed to the environment and configured to engage with a fluid transfer device. The fluid pathway, which extends axially through the NC, is typically sealed by a movable septum at the distal end to prevent entry of pathogens or contaminants into the NC. The pathogens or contaminants could then proceed into the fluid distribution line before entering into the patient's body, causing infection. Alternatively, the pathogens or contaminants could proceed into the fluid source, which would result in contamination. Disinfection of the septum before attaching the fluid source to the NC is crucial for reducing the rate of preventable infections in medical care facilities.

### SUMMARY OF THE INVENTION

Novel aspects of the present disclosure are directed to a sealing device for sealing an opening of an apparatus. In a non-limiting embodiment, the apparatus can be a needleless connector (NC). The sealing device has a head that defines a cavity and a neck that defines a passage disposed between the cavity at a first end of the neck and an aperture at a second end of the neck. The aperture is configured to collapse and expand, having a first diameter in a collapsed configuration and a second diameter in an expanded configuration. The interior surface of the neck includes an engagement interface that is configured to releasably engage with a spacer. When the engagement interface is engaged with the spacer, the neck is in the expanded configuration.

Novel aspects of the present disclosure are also directed to a method of manufacturing a sealing device for sealing an opening. The method includes the step of forming the frame of the sealing device. The sealing device includes a head that defines a cavity and a neck that defines a passage disposed between the cavity at a first end of the neck and an aperture at a second end of the neck. The aperture has a first diameter when the neck is in a collapsed configuration and a second diameter when the neck is in an expanded configuration. The second diameter is larger than the first diameter. An interior surface of the neck includes an engagement interface configured to releasably engage a spacer that maintains the neck in the expanded configuration. The method further includes the step of forming a boundary layer coupled to the frame. The boundary layer encloses the cavity and the neck and defines the aperture at the second end of the neck. The method further includes the step of inserting a spacer into the passage of the neck to engage the engagement interface to cause the neck to transition from the collapsed configuration to the expanded configuration.

Novel aspects of the present disclosure are also directed to a method of using a sealing device configured to seal an opening of an apparatus. The method includes the step of obtaining the sealing device with a neck in an expanded configuration. The sealing device also includes a head that defines a cavity. The neck defines a passage disposed between the cavity at a first end of the neck and an aperture at a second end of the neck. The sealing device further includes a spacer releasably engaged to an engagement interface on an interior surface of the neck to maintain the neck in the expanded configuration. The aperture has a first diameter when the neck is in a collapsed configuration and a second diameter when the neck is in the expanded configuration, the second diameter being greater than the first diameter. The method further includes the steps of aligning the aperture of the sealing device with the opening of the apparatus so that the aperture and the opening are substantially coaxial, providing an insertion force to introduce an end portion of the apparatus housing the opening into the passage of neck, and maintaining the insertion force until the end portion of the apparatus is disposed within the cavity.

Other aspects, embodiments and features of the invention will become apparent from the following detailed description of the invention when considered in conjunction with the accompanying figures. In the figures, each identical, or substantially similar component that is illustrated in various figures is represented by a single numeral or notation. For purposes of clarity, not every component is labeled in every figure. Nor is every component of each embodiment of the invention shown where illustration is not necessary to allow those of ordinary skill in the art to understand the invention.

### BRIEF DESCRIPTION OF THE FIGURES

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying figures, wherein:

FIG. 1 is a perspective view of an exemplary needleless connector;

FIG. 2 is a partial cutaway side view of another exemplary needleless connector;

FIG. 3A is an exploded view of an exemplary sealing device;

FIG. 3B is an exploded cross-section view of the exemplary sealing device in FIG. 3A;

FIG. 3C is an elevation view of an exemplary sealing device and needleless connector;

FIG. 3D is a cross-sectional elevation view of the exemplary sealing device and needleless connector in FIG. 3C;

FIG. 3E is another elevation view of an exemplary sealing device and needleless connector;

FIG. 3F is a cross-sectional elevation view of the exemplary sealing device and needleless connector in FIG. 3E;

FIG. 3G is another elevation view of an exemplary sealing device and needleless connector;

FIG. 3H is cross-sectional elevation view of the exemplary sealing device and needleless connector in FIG. 3G;

FIG. 4A is an exploded view of another exemplary sealing device;

FIG. 4B is an exploded cross-section view of the exemplary sealing device in FIG. 4A;

FIG. 5 is a flowchart of a process for manufacturing a sealing device in accordance with an illustrative embodiment; and

FIG. 6 is a flowchart of a process for using a novel sealing device in accordance with an illustrative embodiment.

### DETAILED DESCRIPTION

Needleless connectors have been identified as a cause of catheter-related bloodstream infection (CRBSI). Inadequate disinfection of NCs allows pathogens to enter a patient's bloodstream, resulting in an expensive and time-consuming road to recovery. The average costs for treating CBRSI is about $48,000, with an increase in the length of stay (LoS) at the hospital by about 7 days. Patients suffering from CBRSI are 5 times more likely to be readmitted to the hospital and experience a 10% increase in mortality rate.

To combat CBRSI, specific disinfection guidelines have been implemented. For example, the current guidelines for engaging needleless connectors (NCs) are as follows: for every engagement, scrub the surface of the NC with an alcohol wipe for 30 seconds, allow the NC to dry for 20 seconds, and then cap the NC when not engaged. In another example, the current guidelines for intravenous (IV) line access with a regular cap are as follows: remove cap, disinfect the exposed surfaces of the needleless connector, connect syringe with saline to check patency, disconnect the syringe, clean the needleless connector again, connect syringe or IV set with medication and deliver, disconnect the syringe or IV, clean the needleless connector again, connect syringe with saline to flush, and disconnect and place a new cap on the needleless connector.

These current disinfection guidelines are complex. Compliance with these guidelines can vary due to subjective interpretation of the steps and due to events occurring within specific medical settings. For example, the manual disinfection with the alcohol wipe can include multiple steps over multiple interfaces. Time constraints are sometimes not followed. Additionally, single-use caps are sometimes reused, particularly when replacement caps are not available. Sometimes caps are improperly attached or not used at all. Even conventional single-use disinfection caps that purport to eliminate the need to disinfect the NC prior to use suffer from user error. For example, some medical care providers continue to disinfect the NC because they do not know if it was previously attached correctly, or if it was removed and reused. Thus, novel aspects of the present disclosure recognize the need for a sealing device that prevents contamination of the NC during storage, disinfects the NC prior to use, and self-modifies to prevent reuse.

While the various embodiments described in this disclosure depict use of the sealing device with needleless connectors, the illustrative use should be deemed exemplary and non-limiting. The sealing devices described herein can be applied equally to connector heads, fittings, or other openings for vessels and fluid conduits in the medical field, as well as in non-medical industries.

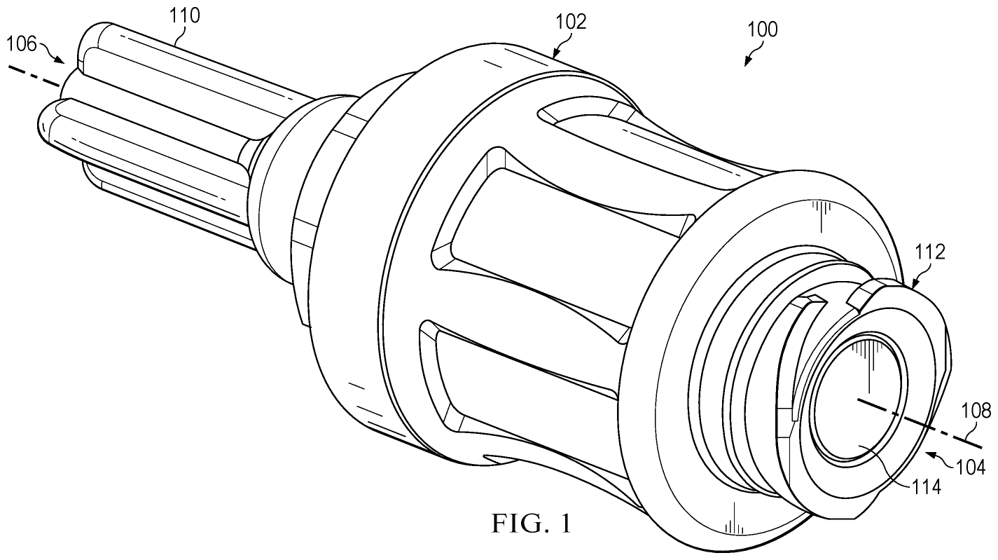

FIG. 1 is a perspective view of an exemplary needleless connector. When attached to a terminal end of a fluid conduit, such as an IV or catheter, the NC 100 selectively seals the fluid conduit to prevent ingress of pathogens and contaminants when not actively in use and permits infusion or aspiration of fluids as required.

The NC 100 includes a housing 102 having a distal end 104 and a proximal end 106. The NC 100 defines a fluid pathway between the distal end 104 and the proximal end 106, coinciding with the axis 108. A tail 110 at the proximal end 106 is configured to engage with a tube (not shown). In this example in FIG. 1, the tail 110 is a narrow, elongated structure configured to be frictionally fit inside of a receiver, such as the terminal end of a tube. A head (or a hub) 112 projects outwardly from the housing 102 at the distal end 104 and is configured to be removably engaged with a receiving end of a fluid transfer device (not shown), such as a syringe, or a fluid dispenser, such as an IV bag or vial (also not shown). In this example in FIG. 1, a threaded interface is disposed on the exterior surface of the head 112 of the NC 100, which is configured to engage a threaded, interior sidewall of the fluid transfer device. In a non-limiting embodiment, the threaded interface of NC 100 is a luer lock fitting.

To prevent the ingress of pathogens into the attached tube via the NC 100, the fluid pathway can be sealed by a movable septum 114 that is partially exposed at distal end 104 of the NC. In one embodiment, the movable septum 114 is an exposed surface of a compressible valve housed within the housing 102. When the compressible valve is exposed to a compression force, the septum 114 disengages from the distal end 104 of the NC 100 to expose an opening that allows fluid to pass from a fluid dispenser through the NC 100 and into the attached tube. The compression force is generally applied to the septum 114 by attachment of a fluid transfer device or fluid source to the head 112 of the NC 100, causing the fluid transfer device or fluid source to engage with the septum 114, unsealing the septum 114 from the distal end 104 of the NC 100.

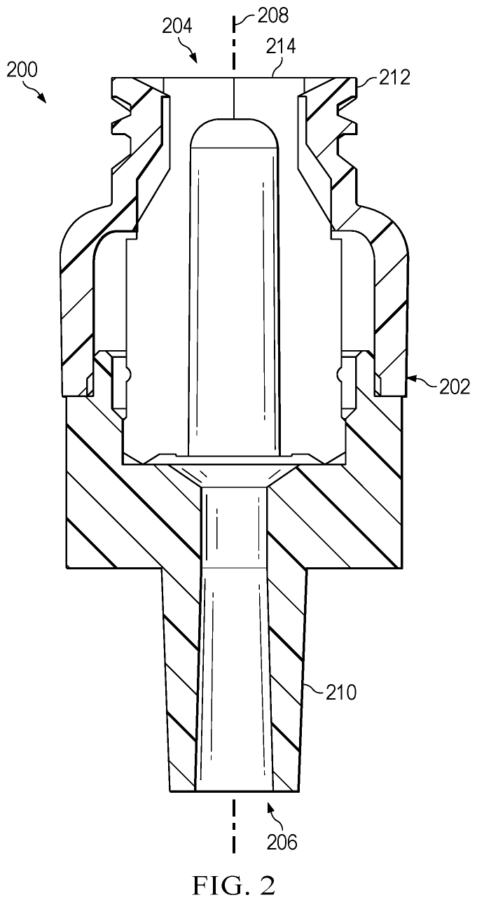

Referring to FIG. 2, a partial cross-sectional view of another exemplary needleless connector is shown. The NC 200 is configured to attach to and selectively seal containers of liquid, e.g., vials, or fluid conduits, such as IVs, to prevent ingress of pathogens when not in use. The NC 200 can permit infusion or aspiration of fluids as required.

The NC 200 includes a housing 202 having a distal end 204 and a proximal end 206. The NC 200 defines a fluid pathway between the distal end 204 and the proximal end 206, coinciding with the axis 208. A tail 210 at the proximal end 206 of the NC 200 is configured to be engaged with a fluid conduit, such as an IV (not shown), or fluid source, such as a container (not shown). A head 212 projects outwardly from the housing 202 at the distal end 204 and is configured to be removably engaged with a receiving end of a fluid transfer device (not shown), such as a syringe. In this example in FIG. 2, a threaded interface is disposed on the exterior surface of the head 212 of the NC 200, which is configured to engage a threaded, interior sidewall of the fluid transfer device. In a non-limiting embodiment, the threaded interface of NC 200 is a luer lock fitting.

To prevent the ingress of pathogens into the attached tube via the NC 200, the fluid pathway can be sealed by a movable septum 214 that is partially exposed at distal end 204 of the NC. In one embodiment, the movable septum 214 is an exposed surface of a compressible valve housed within the housing 202. When the compressible valve is exposed to a compression force, the septum 214 disengages from the distal end 204 of the NC 200 to expose an opening that allows fluid to pass from a fluid dispenser through the NC 200 and into the attached tube. The compression force is generally applied to the septum 214 by attachment of a fluid transfer device to the head 212 of the NC 200, causing the fluid transfer device or fluid source to engage with the septum 214, unsealing the septum 214 from the distal end 204 of the NC 200.

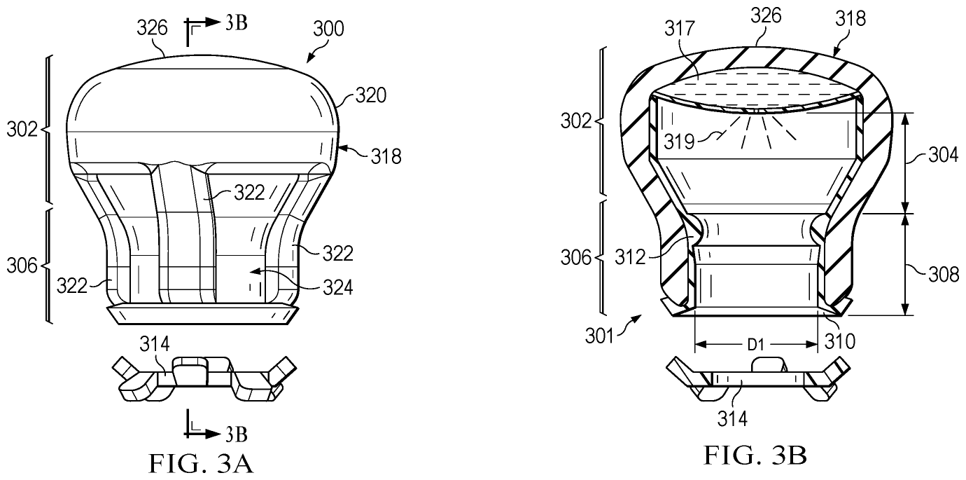

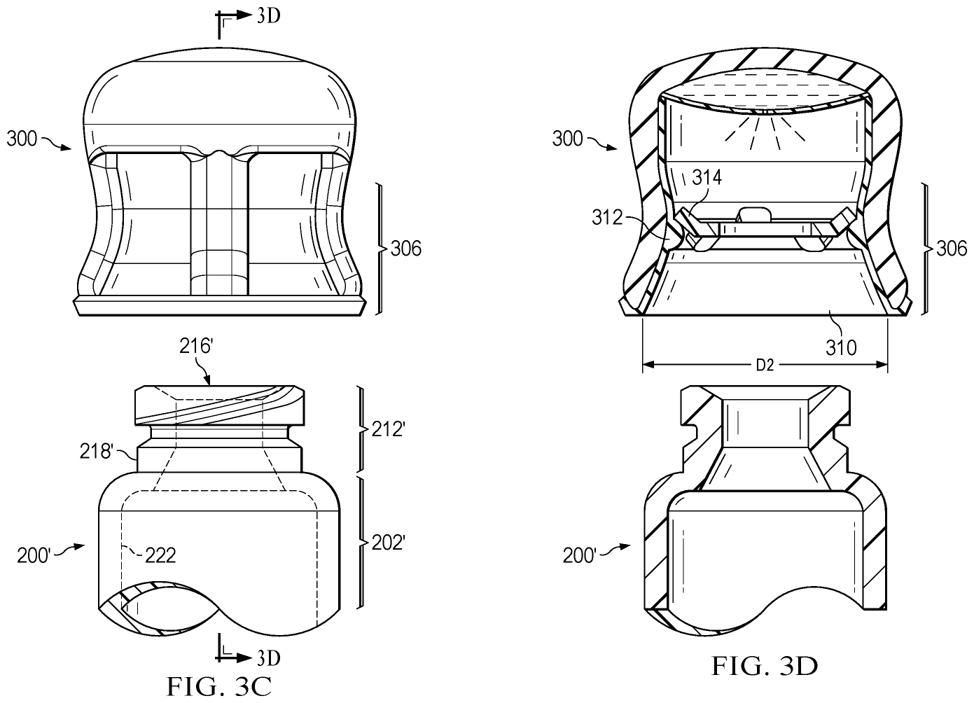

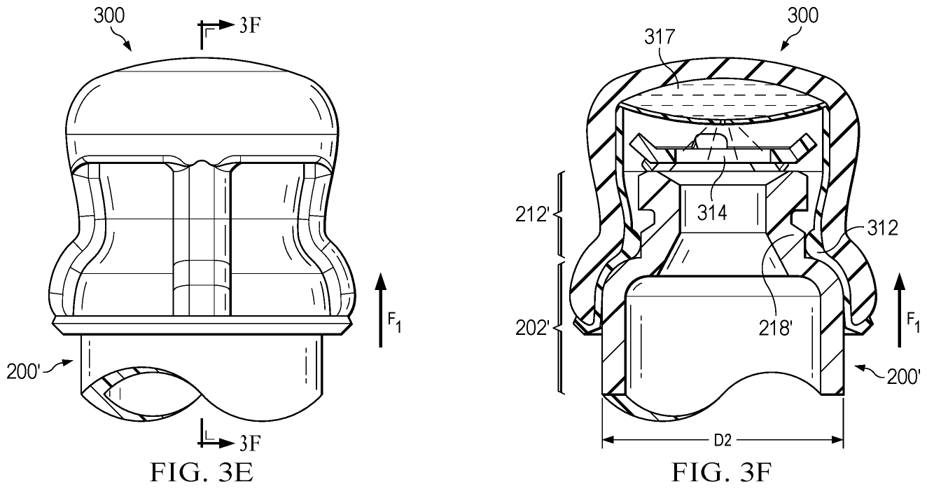

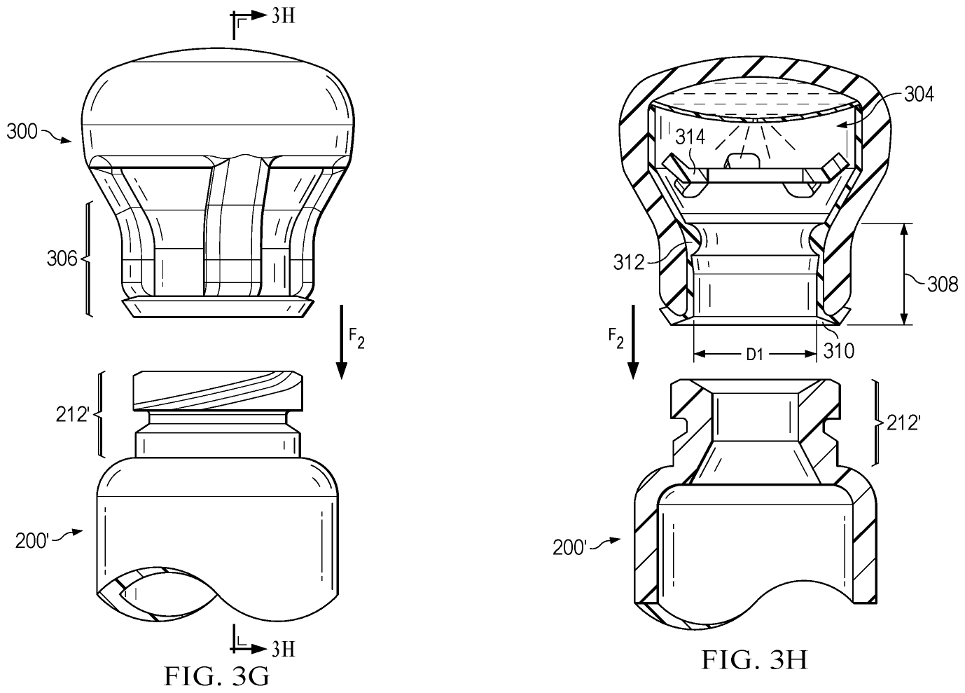

FIGS. 3A-3H are schematic diagrams showing various views of a sealing device 300 usable with the NC 200′ in accordance with an exemplary embodiment. FIGS. 3A and 3G are views of the sealing device 300 in a collapsed configuration, with FIGS. 3B and 3H illustrating cross-sectional views of the sealing device 300 depicted in FIGS. 3A and 3G, respectively. FIGS. 3C and 3E are elevation views of the sealing device 300 in an expanded configuration, with FIGS. 3D and 3F providing cross-sectional views of the sealing device 300 depicted in FIGS. 3D and 3F, respectively. In a non-limiting embodiment, the sealing device 300 is purchased by consumers in the expanded configuration to obviate the need for users to spend the time and efforts to prepare the sealing device 300 for use. When attached to an apparatus, such as a NC, the sealing device 300 seals an opening of the apparatus, thereby preventing fluids from entering or exiting the apparatus.

Referring to FIGS. 3A and 3B, the sealing device 300 includes a head 302 that defines a cavity 304 and a neck 306 that defines a passageway 308. The passageway 308 is disposed between the cavity 304 at a first end of the neck 306 and an aperture 310 at a second end of the neck 306. The neck 306 is configured to expand and contract in a radial direction, which causes the diameter of the aperture 310 to increase and decrease accordingly. In particular, the aperture 310 has a first diameter D1 when the neck 306 is in a collapsed configuration and a second diameter D2 when the neck 306 is in an expanded configuration, as shown in FIG. 3D. In some embodiments, the neck 306 is configured to collapse to the first diameter D1 without the spacer 314 engaging the engagement interface 312 of the neck 306.

The interior surface of the neck 306 includes an engagement interface 312 that is configured to releasably engage with a spacer 314 to maintain the spacer 314 within the neck 306. With the spacer 314 in the neck 306, the sealing device 300 can assume the expanded configuration depicted in FIGS. 3C and 3D. In this illustrative embodiment, the engagement interface 312 is an annulus that projects radially inward. In another embodiment, the engagement interface 312 can be a recessed annulus or a set of recessed slots configured to receive a corresponding engagement feature of the spacer 314. As used herein, the term “set” means one or more. Thus, a set of recessed slots can mean “one recessed slot” or “two or more recessed slots”.

The sealing device 300 may further comprise a disinfectant applicator 317 disposed within the cavity 304 and configured to apply a disinfectant 319 to the hub of a NC. In the sealing device 300 depicted in FIG. 3, the disinfectant applicator 317 is an absorbent material soaked with disinfectant that can be applied to the hub of the NC once the NC has been inserted fully into the cavity 304. The disinfectant applicator 317 may comprise a foam material lining a portion of the inside surface of the cavity 304. The foam material retains the disinfectant 319 via absorption or any other liquid retaining means known in the art. When the hub of the NC is inserted into the cavity 304 of the sealing device 300, the hub compresses the foam material, causing the foam material to release the disinfectant 319 onto the hub.

The sealing device 300 includes a frame 318 that comprises an annular region 320 that at least partially encircles the cavity 304 of the head 302, and a solid crown 326. The frame 318 further comprises a set of arms 322 that are flexibly coupled with the annular region 320 and extend toward the second end of the neck 306. The set of arms 322 provide a compressive force directed radially inward so that the sealing device 300 can assume the collapsed configuration in the absence of spacer 314 inserted within the neck. The flexibility of the set of arms 322 allows the neck 306 to expand sufficiently to receive the hub of a NC.

The sealing device 300 further includes a boundary layer 324 coupled to the frame 320 that encloses the head 302 and the neck 306. The boundary layer 324 defines the aperture 310 at the second end of the neck 306. In the depicted embodiment, the frame 320 encircles the boundary layer 324, but in other embodiments, the frame 320 can be encircled by the boundary layer 324 or the frame 320 can be integrated within the boundary layer 324. The boundary layer may comprise an elastic material. For example, the boundary layer may comprise an overmolded thermoplastic elastomer such as but not limited to styrenic block copolymers, olefinic thermoplastic elastomers, thermoplastic vulcanizates, thermoplastic polyurethanes, thermoplastic copolyester, thermoplastic polyamides, or any other thermoplastic elastomer known in the art.

Referring to FIGS. 3C and 3D, the sealing device is shown in the expanded configuration with the aperture 310 expanded to a second diameter D2. The sealing device 300 can achieve the expanded configuration when the spacer 314 is engaged with the engagement interface 312. When the sealing device 300 is in the expanded configuration, the neck 306 can receive a NC 200′ into cavity 304 (shown in FIG. 3F). Thus, D2 is larger than a diameter of the hub of the NC 200′.

With continued reference to FIGS. 3C and 3D, the NC 200′ includes a housing 202′ having a distal end and a proximal end. The housing 202′ defines an internal chamber 222′. A hub 212′ projects outwardly from the distal end of the housing 202′ and is configured to be removably engaged with the sealing device 300. The hub 212′ includes an opening 216′ of a fluid conduit that extends through the hub 212′ and is in fluid communication with the internal chamber 222′. The hub 212′ may further include a base 218′ positioned at the distal end of the housing 202′.

With reference to FIGS. 3E and 3F, the NC 200′ is sealed within the cavity of sealing device 300. Upon insertion, the spacer 314 is dislodged from the engagement interface 312 by the hub 212′ applying an insertion force F1 on the spacer 314 in the axial direction from the aperture towards the cavity. The spacer 314 is conveyed from the neck 306 into the cavity of the head 302. With the hub 212′ fully inserted into the cavity, the neck 306 of the sealing device 300 forms a seal around the NC 200′. In a non-limiting embodiment, the engagement interface 312 abuts the distal end of the housing 202′, thereby forming a seal between the cavity 304 and the passageway of the neck 306. The engagement interface 312 may comprise a flexible material and have a diameter that is smaller than the exterior diameter of the hub 212′. In such a configuration, the engagement interface 312 forms a tighter seal between the cavity and the passageway of the neck 306. In some embodiments, the engagement surface 312 and the hub 212′ form a hermetic seal between the cavity and the passageway.

With continued reference to FIG. 3F, the disinfectant applicator 317 may apply the disinfectant 319 to the hub 212′ of the NC 200′ in response to an insertion force that causes compression of the disinfectant applicator 317 to release the disinfectant 319. In configurations where the spacer 314 is ring-shaped, the disinfectant applicator 317 may engage with the top surface of the hub 212′ to apply the disinfectant 319. For example, a ring-shaped spacer 314 may have a diameter that is substantially the same size (i.e., within 5% to 10%) of the external diameter of the hub 212′, which allows the disinfectant applicator 317 to apply disinfectant 319 to the top surface of the hub 212′. It is advantageous for the sealing device 300 to apply disinfectant 319 to a large area of the top surface of the hub 212′ because it reduces and/or eliminates the need to disinfect the hub 212′ after withdrawing the NC 200′ from the sealing device 300.

Referring to FIGS. 3G and 3H, when the NC 200′ is ready for use, it is disengaged from the sealing device 300 by applying a pulling force F2 on the NC 200′ from the sealing device 300. As the NC 200′ is disengaged from the sealing device 300, the hub 212′ is withdrawn from the cavity 304 and through the aperture 310 via the passageway 308 of the neck 306. As the hub 212′ is withdrawn from the cavity 304, the reduced diameter of the neck 306 prevents the spacer 314 from exiting the cavity 304, thereby trapping the spacer 314 inside of the cavity 304. After the hub 212′ exits through the aperture 310 of the sealing device 300, the neck 306 returns to its collapsed configuration. When the sealing device 300 is in the collapsed configuration, the first diameter D1 of the aperture 310 has a smaller diameter than the external diameter of the hub 212′ of the NC 200′, which prevents the hub 212′ from being reinserted into the sealing device 300. An advantage of preventing subsequent insertion of a NC into the sealing device 300 is that it prevents reuse of the sealing device 300 which may lead to contamination.

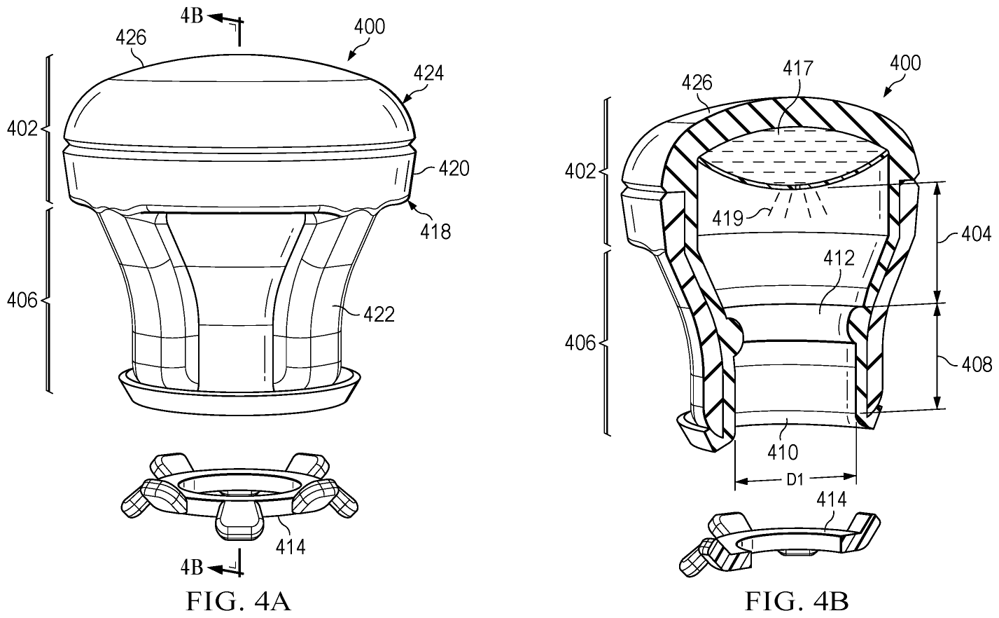

With reference to FIGS. 4A and 4B, an alternative embodiment of a sealing device 400 is depicted. Similar to the previous embodiments described herein, the sealing device 400 comprises a head 402 that defines a cavity 404 and a neck 406 that defines a passageway 408. The neck 406 is configured to expand and contract in a radial direction, which causes the diameter of the aperture 410 to increase and decrease accordingly. In particular, the aperture 410 has a first diameter D1 when the neck 406 is in a collapsed configuration and a second diameter D2 when the neck 406 is in an expanded configuration. In some embodiments, the neck 406 is configured to collapse to the first diameter D1 without the spacer 414 engaging the engagement interface 412 of the neck 406.

The interior surface of the neck 406 includes an engagement interface 412 that is configured to releasably engage with a spacer 414 to maintain the spacer 414 within the neck 406. With the spacer 414 in the neck 406, the sealing device 400 can assume the expanded configuration. In this illustrative embodiment, the engagement interface 412 is an annulus that projects radially inward. In another embodiment, the engagement interface 412 can be a recessed annulus or a set of recessed slots configured to receive a corresponding engagement feature of the spacer 414.

The sealing device 400 includes a frame 418 that comprises an annular region 420 that at least partially encircles the cavity 404 of the head 402. The frame 418 further comprises a set of arms 422 that are flexibly coupled with the annular region 420 and extend toward the second end of the neck 406. The set of arms 422 provide a compressive force directed radially inward so that the sealing device 400 can assume the collapsed configuration in the absence of spacer 414 inserted within the neck 406. The flexibility of the set of arms 422 allows the neck 306 to expand sufficiently to receive the hub of a NC.

The sealing device 400 further includes a boundary layer 424 coupled to the frame 420 that encloses the head 402 and the neck 406. The boundary layer 424 defines the aperture 410 at the second end of the neck 406. In the depicted embodiment, the annular region 420 encircles a circumferential portion of the head 402, with the boundary layer 424 exposed at the crown 426.

The sealing device 400 may further comprise a disinfectant applicator 417 disposed within the cavity 404 and configured to apply a disinfectant 419 to the hub of a NC. In the sealing device 400 depicted in FIG. 4B, the disinfectant applicator 417 is an absorbent material soaked with disinfectant that can be applied to the hub of the NC once the NC has been inserted fully into the cavity 404. The disinfectant applicator 417 may comprise a foam material lining a portion of the inside surface of the cavity 404. The foam material retains the disinfectant 419 via absorption or any other liquid retaining means known in the art. When the hub of the NC is inserted into the cavity 404 of the sealing device 400, the hub compresses the foam material, causing the foam material to release the disinfectant 419 onto the hub. Additionally, a compressive force applied to the crown 426 can cause the disinfectant applicator 417 to engage with the hub of the NC to scrub the hub and apply disinfectant 419.

With continued reference to FIGS. 4A and 4B, it may be advantageous to configure the crown 426 of the head 402 to be enclosed by the boundary layer 424 to facilitate disinfection of the NC. A boundary layer 424 comprised of an elastic material would enable a user to easily apply the disinfectant 419 to the hub by pressing down on the top of the crown 426. As the crown 426 is pressed into the cavity 404, the disinfectant applicator 417 compresses against the hub (not illustrated). As the disinfectant applicator 417 compresses, the disinfectant 419 is displaced and expelled onto the hub. Thus, in configurations where the hub compresses the disinfectant applicator 417 upon insertion into the cavity 404, further compressing the disinfectant applicator 417 via the crown 426 ensures an effective amount of disinfectant 419 is applied to the hub.

In accordance with any of the embodiments described herein, the frame and/or boundary layer may comprise a material having a polymer, a metal, an alloy, or any combination thereof. In some embodiments, the frame comprises a material having a larger elastic modulus than the boundary layer (i.e., the frame is more rigid than the boundary layer). An advantage of the frame comprising a rigid material is that the arms allow for the expansion and contraction of the neck.

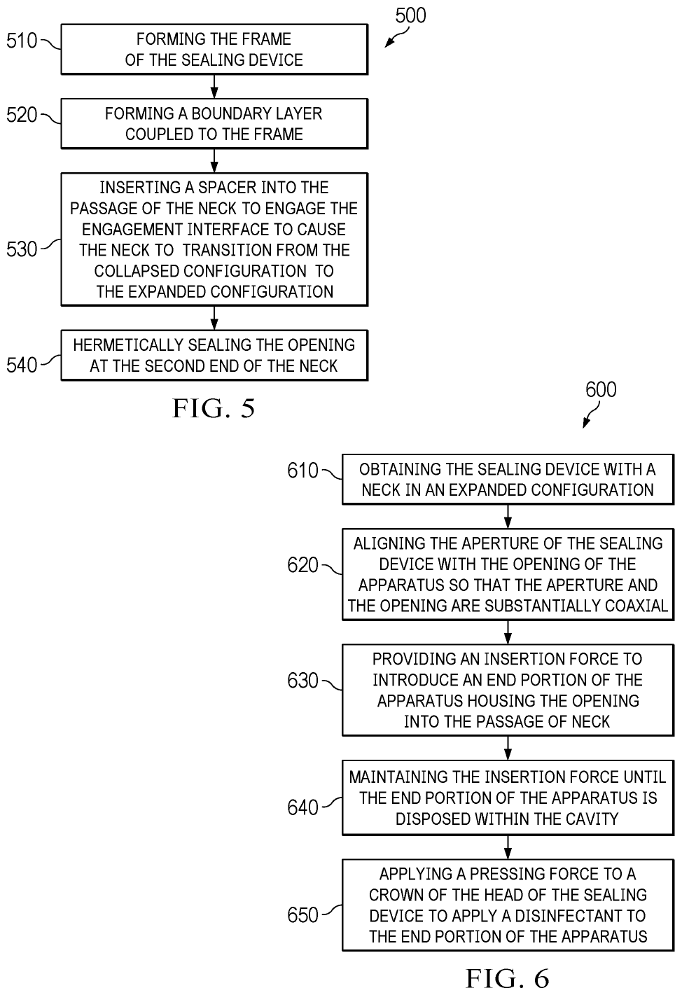

FIG. 5 is a flowchart 500 of a process for manufacturing a sealing device according to an illustrative embodiment. The flowchart 500 begins at step 510 by forming the frame of the sealing device. The sealing device includes a head that defines a cavity, and a neck that defines a passage disposed between the cavity at a first end of the neck and an aperture at a second end of the neck. The aperture has a first diameter when the neck is in a collapsed configuration and a second diameter when the neck is in an expanded configuration, with the second diameter being greater than the first diameter. The interior surface of the neck includes an engagement interface that is configured to releasably engage a spacer that maintains the neck in the expanded configuration.

With continued reference to FIG. 5, in step 520, a boundary layer is formed and coupled to the frame. The boundary layer encloses the cavity and the neck and defines the aperture at the second end of the neck. In some embodiments, the boundary layer is coupled to the frame by overmolding the boundary onto the frame. In step 530, a spacer is inserted into the passage of the neck and engages the engagement interface, thereby causing the neck to transition from the collapsed configuration to the expanded configuration. In some embodiments, a disinfectant applicator may be inserted through the passage of the neck and into the cavity before the spacer is inserted into the passage of the neck. In an optional embodiment, flowchart 500 includes step 540 where the opening end of the neck is hermetically sealed.

FIG. 6 is a flowchart 600 of a process for using a sealing device configured to seal an opening of an apparatus according to an illustrative embodiment. The flowchart 600 begins at step 610 by obtaining the sealing device having a neck in an expanded configuration. The sealing device includes a head that defines a cavity. The neck defines a passage disposed between the cavity at a first end of the neck and an aperture at a second end of the neck. The sealing device further includes a spacer releasably engaged to an engagement interface on an interior surface of the neck to maintain the neck in the expanded configuration. The aperture has a first diameter when the neck is in a collapsed configuration and a second diameter when the neck is in the expanded configuration, with the second diameter being greater than the first diameter.

With continued reference to FIG. 6, in step 620, the aperture of the sealing device is aligned with the opening of the apparatus so that the aperture and the opening are substantially coaxial. In step 630, an insertion force is provided to introduce an end portion of the apparatus housing to the opening into the passage of neck. In step 640, the insertion force is maintained until the end portion of the apparatus is disposed within the cavity. The insertion force may cause the end portion of the apparatus to contact the spacer. The insertion force disengages the spacer from the engagement interface, thereby causing the neck to attain the collapsed configuration. In step 650, a pressing force is applied to a crown of the head of the sealing device to apply a disinfectant to the end portion of the apparatus.

Although embodiments of the invention have been described with reference to several elements, any element described in the embodiments described herein are exemplary and can be omitted, substituted, added, combined, or rearranged as applicable to form new embodiments. A skilled person, upon reading the present specification, would recognize that such additional embodiments are effectively disclosed herein. For example, where this disclosure describes characteristics, structure, size, shape, arrangement, or composition for an element or process for making or using an element or combination of elements, the characteristics, structure, size, shape, arrangement, or composition can also be incorporated into any other element or combination of elements, or process for making or using an element or combination of elements described herein to provide additional embodiments.

Additionally, where an embodiment is described herein as comprising some element or group of elements, additional embodiments can consist essentially of or consist of the element or group of elements. Also, although the open-ended term “comprises” is generally used herein, additional embodiments can be formed by substituting the terms “consisting essentially of” or “consisting of”

While this invention has been particularly shown and described with reference to preferred embodiments, it will be understood by those skilled in the art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A sealing device for sealing an opening of an apparatus, the sealing device comprising:
a head that defines a cavity;
a neck that defines a passage disposed between the cavity at a first end of the neck and an aperture at a second end of the neck, wherein:
the aperture has a first diameter when the neck is in a collapsed configuration and the aperture has a second diameter when the neck is in an expanded configuration, the second diameter being greater than the first diameter, and
an interior surface of the neck includes an engagement interface configured to releasably engage a spacer that maintains the neck in the expanded configuration.

2. The sealing device of claim 1, wherein:
the apparatus is a needleless connector comprising a hub,
the first diameter of the aperture is smaller than an external diameter of the hub of the needleless connector to prevent insertion of the hub into the passageway when the neck is in the collapsed configuration, and
the second diameter is greater than the external diameter of the hub of the needleless connector to permit insertion of the hub into the passage when the neck is in the expanded configuration.

3. The sealing device of claim 1, wherein the spacer is coupled to the engagement interface to maintain the neck in the expanded configuration.

4. The sealing device of claim 3, wherein:
the engagement interface is an annulus that extends radially inward, and
the spacer frictionally engages the annulus to maintain the neck in the expanded configuration.

5. The sealing device of claim 3, wherein:
the engagement interface is a recess, and
the spacer frictionally engages with the recess to maintain the neck in the expanded configuration.

6. The sealing device of claim 3, wherein the cavity is dimensioned to accommodate the spacer and an end portion of the apparatus that houses the opening of the apparatus.

7. The sealing device of claim 1, wherein the engagement interface is configured to release the spacer in response to an insertion force imparted in an axial direction from the aperture at the second end of the neck towards the cavity at the first end of the neck.

8. The sealing device of claim 7, wherein the insertion force is imparted onto the spacer by the apparatus that houses the opening.

9. The sealing device of claim 1, further comprising:
a frame that includes an annular region that at least partially encircles the cavity and a set of arms flexibly coupled with the annular region, wherein the set of arms extends toward the second end of the neck; and
a boundary layer coupled to the frame to enclose the cavity and the neck, wherein the boundary layer defines the aperture at the second end of the neck.

10. The sealing device of claim 9, wherein the frame encloses a portion of the head so that the boundary layer is exposed at a crown of the head.

11. The sealing device of claim 10, further comprising a disinfectant applicator within the cavity, wherein the disinfectant applicator is configured to apply disinfectant to the opening of the apparatus in response to a pressing force applied to the crown.

12. The sealing device of claim 11, wherein the spacer is ring-shaped to permit the disinfectant applicator to engage with the opening of the apparatus.

13. The sealing device of claim 9, wherein the boundary layer is an overmolded thermoplastic elastomer.

14. A method of manufacturing a sealing device for sealing an opening, the method comprising:
forming the frame of the sealing device, wherein the sealing device includes:
a head that defines a cavity, and
a neck that defines a passage disposed between the cavity at a first end of the neck and an aperture at a second end of the neck, wherein:
the aperture has a first diameter when the neck is in a collapsed configuration and the aperture has a second diameter when the neck is in an expanded configuration, the second diameter being greater than the first diameter, and
an interior surface of the neck includes an engagement interface configured to releasably engage a spacer that maintains the neck in the expanded configuration;
forming a boundary layer coupled to the frame, wherein:
the boundary layer encloses the cavity and the neck, and
the boundary layer defines the aperture at the second end of the neck; and
inserting a spacer into the passage of the neck to engage the engagement interface to cause the neck to transition from the collapsed configuration to the expanded configuration.

15. The method of claim 14, wherein forming the boundary layer further comprises overmolding the boundary layer onto the frame.

16. The method of claim 14 further comprising inserting a disinfectant applicator through the passage of the neck and into the cavity before inserting the spacer into the passage of the neck.

17. The method of claim 14, further comprising hermetically sealing the opening at the second end of the neck.

18. A method using a sealing device configured to seal an opening of an apparatus, the method comprising:
obtaining the sealing device with a neck in an expanded configuration, wherein the sealing device includes:
a head that defines a cavity, wherein the neck defines a passage disposed between the cavity at a first end of the neck and an aperture at a second end of the neck, and
a spacer releasably engaged to an engagement interface on an interior surface of the neck to maintain the neck in the expanded configuration, wherein:
the aperture has a first diameter when the neck is in a collapsed configuration,
the aperture has a second diameter when the neck is in the expanded configuration, and
the second diameter is greater than the first diameter;
aligning the aperture of the sealing device with the opening of the apparatus so that the aperture and the opening are substantially coaxial;
providing an insertion force to introduce an end portion of the apparatus housing the opening into the passage of neck; and
maintaining the insertion force until the end portion of the apparatus is disposed within the cavity.

19. The method of claim 18, wherein:
the insertion force causes the end portion of the apparatus to contact the spacer; and
the insertion force disengages the spacer from the engagement interface to cause the neck to attain the collapsed configuration.

20. The method of claim 18, further comprising applying a pressing force to a crown of the head of the sealing device to apply a disinfectant to the end portion of the apparatus.
